(19) United States
(12) Patent Application Publication
(10) Pub. No.: US 2004/0182584 A1
(43) Pub. Date: Sep. 23, 2004

(54) Fire port with frangible crosshair

(76) Inventor: David M. Thompson, Bricktown, NJ (US)

Correspondence Address: Andrew W. Ludy, 17 Sherwood Way, Landing, NJ, 07850, US

(21) Appl. No.: 10/390,104

(22) Filed: Mar. 17, 2003

(51) Int. Cl. A62C 2/00; A61L 9/04; A62C 3/00; A24F 25/00

(52) U.S. Cl. 169/46

(57) ABSTRACT

A fire access port is mounted on a flammable material compartment juxtaposed with an access hole. A fire extinguisher nozzle is inserted through the fire port to suppress a fire from outside the compartment. The fire port comprises a diaphragm of flexible, resilient material having a predetermined thickness extending between opposite primary and secondary surfaces. First and second transverse grooves extend across the diaphragm primary surface. The grooves have a maximum depth less than the diaphragm thickness, and a floor at the maximum depth, forming first and second frangible webs between each groove floor and the secondary surface. The frangible webs will rupture as the fire extinguisher nozzle is inserted through the diaphragm.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

This invention relates to the field of fire extinguishing equipment, and more particularly to a fire access port for a flammable material compartment through which fire extinguishing equipment may be inserted to suppress a fire from outside the compartment.

Flammable materials are commonly stored or processed in a compartment, locker, container, or shed. Volatiles such as paints, solvents, or chemicals, contained within such a compartment, are subject to accidental fires due to various causes. Typical causes can be spontaneous combustion, lightning strike, electrical short-circuit resulting in overheated wiring, or a carelessly discarded cigarette. Similarly, a vehicle engine, particularly on a boat, is typically housed within a compartment or box. The compartment isolates passengers from the noise, fumes, moving parts, heat, and fire hazard of the engine, and protects the engine from the weather. Despite precautions, engine fires sometimes break out. A fuel leak will spray flammable fuel on a hot engine, resulting in a fire.

Opening the flammable material compartment to fight the fire exposes the operator to heat, flames, and smoke. Opening the flammable material compartment also admits oxygen to feed the fire. A common expedient to fighting such a fire is to install a fire access port on the flammable material compartment. The fire access port typically is a disc of resilient material cut to form a flap or valve. Mounted on the compartment, the fire port is normally in a closed or sealed state. In the event of a fire, a fire extinguisher nozzle is thrust through the fire port, and the fire suppressant chemical is released, without opening the compartment.

Fire access ports are known and have assumed a number of embodiments in the past. Some examples of access ports in the prior art are revealed in the following U.S. Pat. Nos.

Thompson, U.S. Pat. No. 5,511,622, illustrates a fire port valve having a resilient flap, or stop, attached at the top, which is normally closed. The stop flexes inward, at the attachment, when the nozzle of a fire extinguisher is inserted through the fire port.

Stary, U.S. Pat. No. 4,047,572, discloses a fire access port with two cross slits through a resilient diaphragm. Pressure at the center will immediately permit the flaps of the diaphragm to yield, admitting the nozzle.

Baldwin, U.S. Pat. No. 3,729,031, shows a liquid dispenser and filling apparatus having a resilient sealing plunger sliding within a barrel. The plunger has a self-sealing cross-slit which is pushed open by a fill member in order to fill the barrel with liquid.

Draben, U.S. Pat. No. 3,354,508, illustrates a charging fitting for plastic molding apparatus. A resilient diaphragm with a cross-slit is pushed open by a nozzle to discharge liquid resin therethrough.

In installing a fire port, of the type described above, in a boat or on an outside locker or shed, it is desirable to seal the port against water entering, especially where the compartment is exposed to the weather. Conversely, it is desirable to seal the port against fumes escaping, especially where an engine compartment is inside a cabin. In none of the above-described inventions is the diaphragm positively sealed. In each case, the slit is completely through the diaphragm, so as to facilitate insertion of the nozzle. Such a slit is made with a knife-edge tool, requiring a secondary operation subsequent to molding, with resultant increased cost.

Accordingly, there is a need to provide a fire port that is sealed positively against water entering the flammable material compartment.

There is a further need to provide a fire port of the type described and that is sealed positively against fumes and noise escaping the flammable material compartment.

There is yet a further need to provide a fire port of the type described and that will readily permit easy and rapid insertion of the fire extinguisher nozzle.

There is a still further need to provide a fire port of the type described and that will be ready to install right from molding, with no secondary operation, and hence can be economically manufactured in large numbers of high quality.

There is another need to provide a fire port of the type described and that is easy to use under emergency conditions in the field.

There is yet another need to provide a fire port of the type described and that is rugged in construction so as to provide reliable performance over an extended service life.

### BRIEF SUMMARY OF THE INVENTION

In accordance with the present invention, there is provided a fire port for a flammable material compartment. The compartment has an access hole through it. The fire port comprises a diaphragm of flexible, resilient material. The diaphragm has a predetermined thickness extending between opposite primary and secondary surfaces. The diaphragm has at least one first groove extending across the primary surface. The first groove has a maximum depth less than the diaphragm thickness, and a floor at the maximum depth. The diaphragm has a first frangible web between the first groove floor and the secondary surface. The diaphragm is juxtaposed with the flammable material compartment access hole.

The diaphragm includes a second groove transverse to the first groove. The second groove extends across the primary surface, and has a floor generally at the same depth as the first groove floor. The diaphragm has a second frangible web between the second groove floor and the secondary surface.

Mounting means are provided for mounting the fire port on the flammable material compartment. Thus, in the event of a fire, a fire extinguisher nozzle will be inserted through the diaphragm, the frangible web will rupture, and the nozzle will enter the flammable material compartment. The fire extinguisher will then be actuated, thereby suppressing the fire.

### BRIEF DESCRIPTION OF THE SEVERAL VIEWS OF THE DRAWING

The invention will be more fully understood, while still further objects and advantages will become apparent, in the following detailed description of preferred embodiments thereof illustrated in the accompanying drawing, in which:

FIG. 1 is a cross-sectional view of a Fire Port With Frangible Crosshair, constructed in accordance with the invention, and mounted in a flammable material compartment, with a fire extinguisher nozzle penetrating the fire port;

FIG. 2 is a top perspective view of the fire port of FIG. 1;

FIG. 3 is a bottom perspective view of the fire port of FIG. 1;

FIG. 4 is a top view of the fire port of FIG. 1;

FIG. 5 is a front cross-sectional view of the fire port of FIG. 1, taken along lines 5-5 of FIG. 4;

FIG. 6 is a front cross-sectional view of the fire port of FIG. 1, taken along lines 6-6 of FIG. 4;

FIG. 7 is an enlarged detail view of FIG. 6, taken at circle 7 of FIG. 6;

FIG. 8 is a top perspective view of another Fire Port With Frangible Crosshair, constructed in accordance with the invention;

FIG. 9 is a bottom perspective view of the fire port of FIG. 8;

FIG. 10 is a top view of the fire port of FIG. 8;

FIG. 11 is a front cross-sectional view of the fire port of FIG. 10, taken along lines 11-11 of FIG. 10;

FIG. 12 is an enlarged detail view of FIG. 11, taken at circle 12 of FIG. 11;

FIG. 13 is a top perspective view of yet another Fire Port With Frangible Crosshair, constructed in accordance with the invention;

FIG. 14 is a bottom perspective view of the fire port of FIG. 13;

FIG. 15 is a top view of the fire port of FIG. 13;

FIG. 16 is a front cross-sectional view of the fire port of FIG. 13, taken along lines 16-16 of FIG. 15; and

FIG. 17 is an enlarged detail view of FIG. 16, taken at circle 17 of FIG. 16.

### DETAILED DESCRIPTION OF THE INVENTION

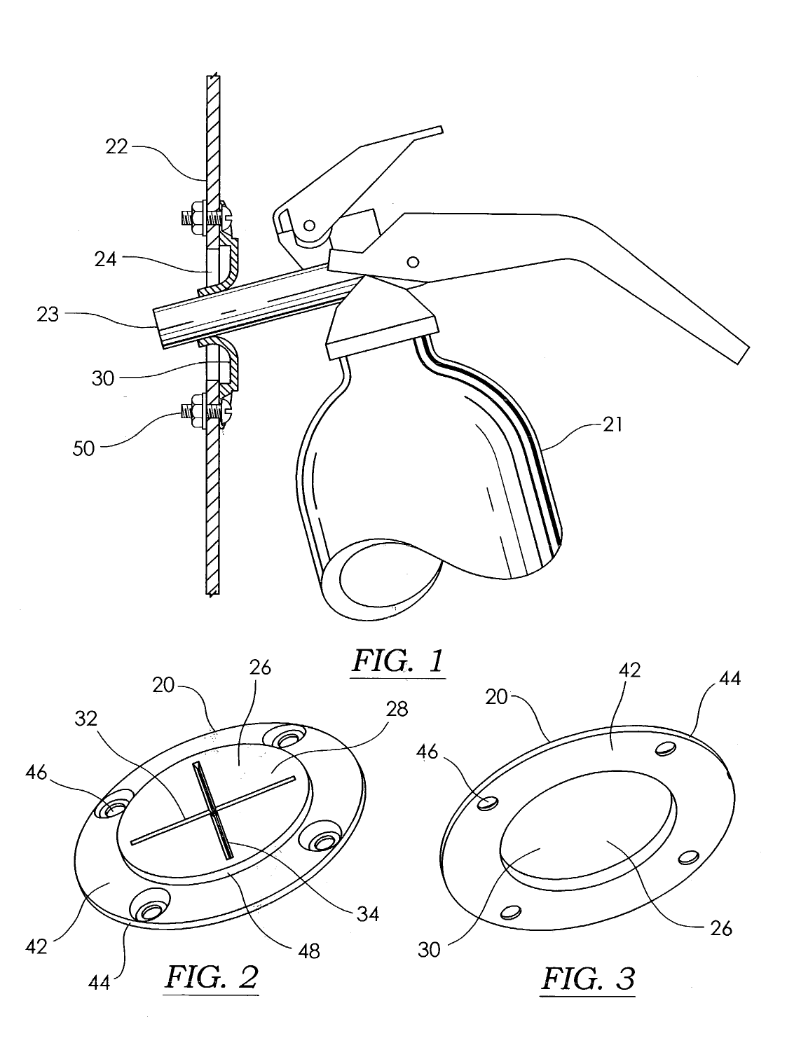

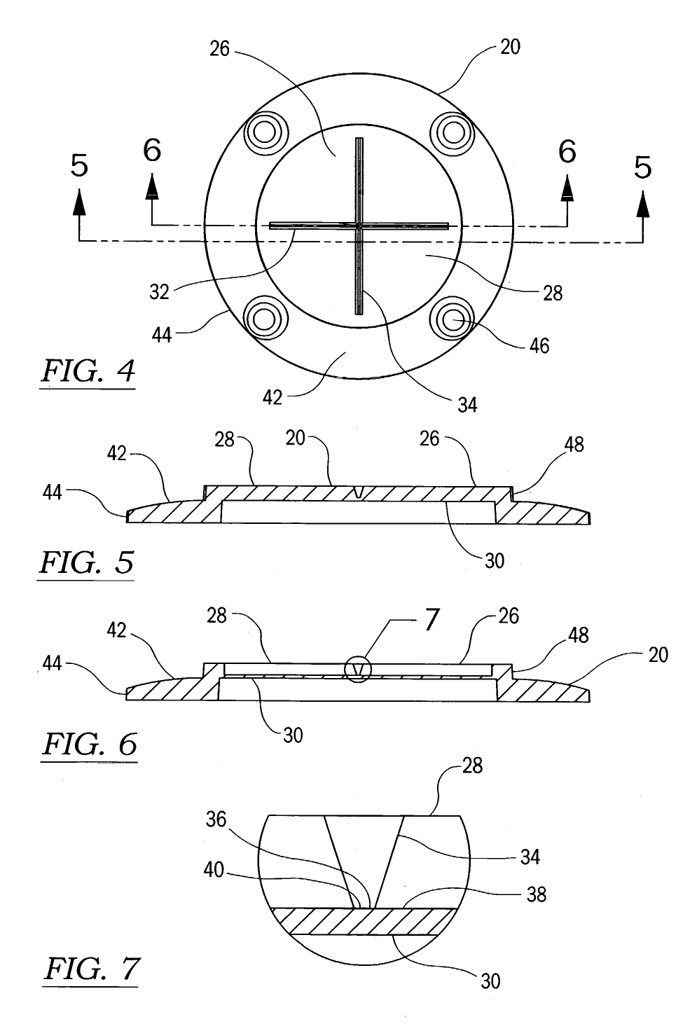

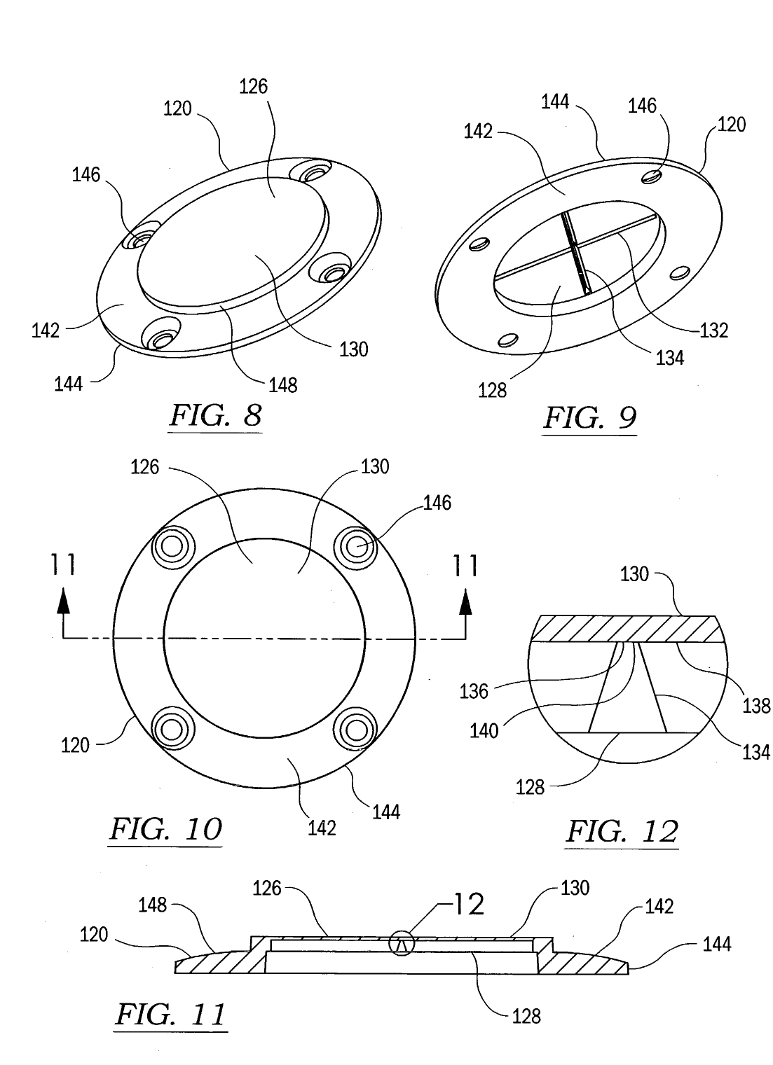

Referring now to the drawing, and especially to FIGS. 1, 2, 3, 4, 5, 6, and 7 thereof, a fire port constructed in accordance with the invention is shown at 20, and is for use with a fire extinguisher 21, with a nozzle 23, as depicted in FIG. 1. The fire port 20 comprises a diaphragm 26, preferably made of PVC, but any flexible, resilient, frangible material can be used. In the preferred embodiment, the diaphragm 26 is circular in shape. The diaphragm 26 has a predetermined thickness, typically 0.065 inch (1.65 mm), but any convenient thickness will work. The diaphragm 26 extends between opposite primary 28 and secondary 30 surfaces. The flammable material compartment 22 has an access hole 24 therethrough. The diaphragm 26 is mounted on the compartment 22, and is juxtaposed with the compartment access hole 24. The primary surface 28 faces outward on the compartment 22. The diaphragm 26 has first 32 and second 34 grooves extending across the primary surface 28. The second groove 34 is transverse to the first groove 32. The first 32 and second 34 grooves have a maximum depth less than the diaphragm thickness. The first 32 and second 34 grooves each have a floor 36 at the maximum depth, as shown in FIG. 7. The diaphragm 26 has a first frangible web 38 between the first groove floor 36 and the secondary surface 30. The diaphragm 26 has a second frangible web 40 between the second groove floor 36 and the secondary surface 30. The first 32 and second 34 grooves taper outward in width from the floor 36 to the primary surface 28, forming a generally V-shaped cross-section, as shown in FIG. 7, to provide draft for molding. The webs 38 and 40 are typically about 0.012 inch (0.3 mm), but this will vary with the thickness of the diaphragm and the material used.

A base member 42 extends from the diaphragm 26 outward to a periphery 44. The base member 42 is typically an annular ring-like structure extending around the diaphragm 26. Mounting means is provided for mounting the fire port 20 on the flammable material compartment 22. Specifically, fire port 20 includes a plurality of mounting holes 46 therethrough, arrayed around the base member 42. A nut and bolt 50 typically extends through each mounting hole 46 and through the compartment 22, although any type of fastener can be used. A shoulder 48 connects the diaphragm 26 to the base member 42. The shoulder 48 extends around the diaphragm 26, to reinforce the diaphragm. Thus, in the event of a fire, the fire extinguisher nozzle 23 will be inserted through the diaphragm 26, the frangible webs 38 and 40 will rupture, the nozzle 23 will enter the flammable material compartment 22, and the fire extinguisher 21 will be actuated, thereby suppressing the fire.

Turning now to FIGS, 8, 9, 10, 11, and 12, another fire port constructed in accordance with the invention is shown at 120. Fire port 120 is similar to the invention of FIG. 2, in that fire port 120 comprises a diaphragm 126, which is circular in shape. The diaphragm 126 has a predetermined thickness, and extends between opposite primary 128 and secondary 130 surfaces. The diaphragm 126 has first 132 and second 134 grooves extending across the primary surface 128. The second groove 134 is transverse to the first groove 132. The first 132 and second 134 grooves have a maximum depth less than the diaphragm thickness. The first 132 and second 134 grooves each have a floor 136 at the maximum depth, as shown in FIG. 12. The diaphragm 126 has a first frangible web 138 between the first groove floor 136 and the secondary surface 130. The diaphragm 126 has a second frangible web 140 between the second groove floor 136 and the secondary surface 130. The first 132 and second 134 grooves taper outward in width from the floor 136 to the primary surface 128, forming a generally V-shaped cross-section, as shown in FIG. 12.

A base member 142 extends from the diaphragm 126 outward to a periphery 144. The base member 142 extends around the diaphragm 126. Fire port 120 includes a plurality of mounting holes 146 therethrough, arrayed around the base member 142. A shoulder 148 extends around the diaphragm 126, and connects the diaphragm 126 to the base member 142.

Fire port 120 differs from the invention of FIG. 2, in that the secondary surface 130 faces outward on the flammable material compartment (not shown). The primary surface 128, having the grooves 132 and 134, faces inward on the compartment.

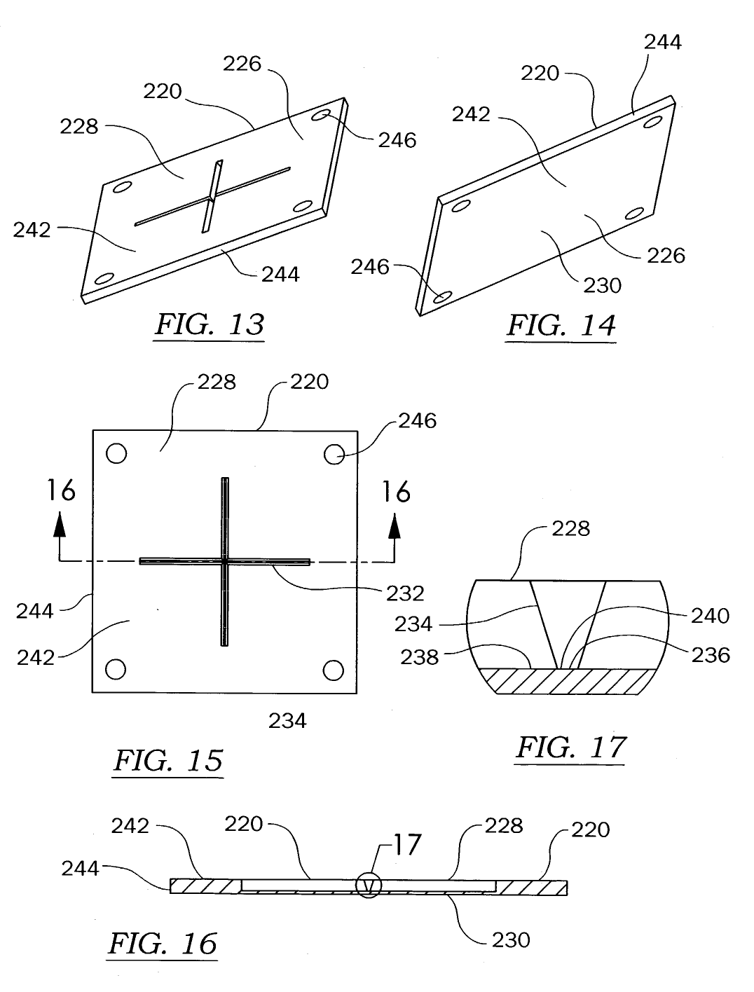

Referring now to FIGS. 13, 14, 15, 16, and 17, another embodiment of the invention is shown at 220. Fire port 220 is similar to the invention of FIG. 2, in that fire port 220 comprises a diaphragm 226. The diaphragm 226 has a predetermined thickness, and extends between opposite primary 228 and secondary 230 surfaces. The diaphragm 226 has first 232 and second 234 grooves extending across the primary surface 228. The second groove 234 is transverse to the first groove 232. The first 232 and second 234 grooves have a maximum depth less than the diaphragm thickness. The first 232 and second 234 grooves each have a floor 236 at the maximum depth, as shown in FIG. 17. The diaphragm 226 has a first frangible web 238 between the first groove floor 236 and the secondary surface 230. The diaphragm 226 has a second frangible web 240 between the second groove floor 236 and the secondary surface 230. The first 232 and second 234 grooves taper outward in width from the floor 236 to the primary surface 228, forming a generally V-shaped cross-section, as shown in FIG. 17. Fire port 220 includes a plurality of mounting holes 246 therethrough.

Fire port 220 differs from the invention of FIG. 2, in that the diaphragm 226 extends outward through a base member 242 to a rectangular periphery 244. The diaphragm 226 is thus unitary with the base member 242. There is no shoulder. The primary surface 228, having the grooves 232 and 234, can face either inward or outward on the flammable material compartment (not shown).

A method is also disclosed for fighting a fire in a flammable material compartment 22 through a fire port 20. The method comprises the steps of molding a diaphragm 26, in the fire port 20, of flexible material to a predetermined thickness, then extending the diaphragm 26 between opposite primary 28 and secondary 30 surfaces. Next, extending a first groove 32 across the primary surface 28, and providing a floor 36 in the first groove 32 at a maximum depth less than the diaphragm thickness, then forming a first frangible web 38 in the diaphragm 26 between the first groove floor 36 and the secondary surface 30. Next, juxtaposing the diaphragm 26 with the compartment access hole 24, and mounting the fire port 20 on the flammable material compartment 22. Next, inserting a fire extinguisher nozzle 23 through the diaphragm 26 into the compartment 22, rupturing the frangible web 38, actuating the fire extinguisher 21, and suppressing the fire.

Further steps comprise extending a second groove 34, transverse to the first groove 32, across the primary surface 28, and providing a floor 36 in the second groove 34 generally at the same depth as the first groove floor 36, then forming a second frangible web 40 in the diaphragm 26 between the second groove floor 36 and the secondary surface 30. Next, forming a plurality of mounting holes 46 through the fire port 20 for mounting the fire port 20 on the compartment 22.

Yet further steps comprise extending a base member 42 from the diaphragm 26 outward to a periphery 44, extending the base member 42 around the diaphragm 26, and disposing the mounting holes 46 within the base member 42. Next, connecting the diaphragm 26 to the base member 42 with a shoulder 48, and extending the shoulder 48 around the diaphragm 26, to reinforce the diaphragm 26 for inserting the nozzle 23 therethrough.

Still further steps comprise tapering the first 32 and second 34 grooves outward in width from the floor 36 to the primary surface 28, forming a generally V-shaped cross-section, to provide draft for molding.

Another step comprises facing the primary surface 28 outward on the flammable material compartment. An alternative step is facing secondary surface 30 outward on the compartment.

Numerous modifications and alternative embodiments of the invention will be apparent to those skilled in the art in view of the foregoing description. Accordingly, this description is to be construed as illustrative only and is for the purpose of teaching those skilled in the art the best mode of carrying out the invention. Details of the structure may be varied substantially without departing from the spirit of the invention and the exclusive use of all modifications that will come within the scope of the appended claims is reserved.

The embodiments of the invention in which an exclusive property or privilege is claimed are defined as follows:

1. A fire port for a flammable material compartment, the compartment having an access hole therethrough, the fire port comprising:
a diaphragm, the diaphragm having a predetermined thickness extending between opposite primary and secondary surfaces, the diaphragm having at least one first groove extending across the primary surface, the first groove having a maximum depth less than the diaphragm thickness, the first groove having a floor at the maximum depth, the diaphragm having a first frangible web between the first groove floor and the secondary surface, the diaphragm being juxtaposed with the compartment access hole; and
mounting means for mounting the fire port on the compartment, so that in the event of a fire, a fire extinguisher nozzle will be inserted through the diaphragm, the frangible web will rupture, the nozzle will enter the compartment, and the fire extinguisher will be actuated, thereby suppressing the fire.

2. The fire port of claim 1, wherein the diaphragm includes a second groove transverse to the first groove, the second groove extending across the primary surface, the second groove having a floor generally at the same depth as the first groove floor, the diaphragm having a second frangible web between the second groove floor and the secondary surface.

3. The fire port of claim 2, wherein the fire port includes a plurality of mounting holes therethrough, for mounting the fire port on the flammable material compartment.

4. The fire port of claim 3, further comprising:
a base member extending from the diaphragm outward to a periphery, the base member extending around the diaphragm, the mounting holes being disposed within the base member; and
a shoulder connecting the diaphragm to the base member, the shoulder extending around the diaphragm, so as to reinforce the diaphragm as the nozzle is inserted therethrough.

5. The fire port of claim 4, wherein the first and second grooves taper outward in width from the floor to the primary surface, forming a generally V-shaped cross-section, to provide draft for molding.

6. The fire port of claim 5, wherein the primary surface faces outward on the flammable material compartment.

7. The fire port of claim 5, wherein the secondary surface faces outward on the flammable material compartment.

8. A fire port for a flammable material compartment, the compartment having an access hole therethrough, the fire port comprising:
a diaphragm of flexible material, the diaphragm having a predetermined thickness, the diaphragm extending between opposite primary and secondary surfaces, the diaphragm having first and second grooves extending across the primary surface, the second groove being transverse to the first groove, the first and second grooves having a maximum depth less than the diaphragm thickness, the first and second grooves each having a floor at the maximum depth, the diaphragm having a first frangible web between the first groove floor and the secondary surface, the diaphragm having a second frangible web between the second groove floor and the secondary surface, the diaphragm being juxtaposed with the compartment access hole;
a base member extending from the diaphragm outward to a periphery, the base member extending around the diaphragm, the base member having a plurality of mounting holes for mounting the fire port on the compartment; and
a shoulder connecting the diaphragm to the base member, the shoulder extending around the diaphragm, to reinforce the diaphragm, so that in the event of a fire, a fire extinguisher nozzle will be inserted through the diaphragm, the frangible webs will rupture, the nozzle will enter the compartment, and the fire extinguisher will be actuated, thereby suppressing the fire.

9. The fire port of claim 8, wherein the first and second grooves taper outward in width from the floor to the primary surface, forming a generally V-shaped cross-section, to provide draft for molding.

10. The fire port of claim 9, wherein the primary surface faces outward on the flammable material compartment.

11. The fire port of claim 9, wherein the secondary surface faces outward on the flammable material compartment.

12. A method for suppressing a fire in a flammable material compartment through a fire port, the compartment having an access hole therethrough, the method comprising the steps of:
molding a diaphragm, in the fire port, of flexible material to a predetermined thickness;
extending the diaphragm between opposite primary and secondary surfaces;
extending a first groove across the primary surface;
providing a floor in the first groove at a maximum depth less than the diaphragm thickness;
forming a first frangible web in the diaphragm between the first groove floor and the secondary surface;
juxtaposing the diaphragm with the compartment access hole;
mounting the fire port on the compartment;
inserting a fire extinguisher nozzle through the diaphragm, into the compartment;
rupturing the frangible web;
actuating the fire extinguisher; and
suppressing the fire.

13. The method of claim 12, further comprising the steps of:
extending a second groove, transverse to the first groove, across the primary surface;
providing a floor in the second groove generally at the same depth as the first groove floor; and
forming a second frangible web in the diaphragm between the second groove floor and the secondary surface.

14. The method of claim 13, further comprising the step of forming a plurality of mounting holes through the fire port for mounting the fire port on the flammable material compartment.

15. The method of claim 14, further comprising the steps of:
extending a base member from the diaphragm outward to a periphery;
extending the base member around the diaphragm;
disposing the mounting holes within the base member; and
connecting the diaphragm to the base member with a shoulder; and
extending the shoulder around the diaphragm, to reinforce the diaphragm for inserting the nozzle therethrough.

16. The method of claim 15, further comprising the step of tapering the first and second grooves outward in width from the floor to the primary surface, forming a generally V-shaped cross-section, to provide draft for molding.

17. The method of claim 16, further comprising the step of facing the primary surface outward on the flammable material compartment.

18. The method of claim 16, further comprising the step of facing secondary surface outward on the flammable material compartment.
